(19) United States
(12) Patent Application Publication
(10) Pub. No.: US 2016/0262692 A1
(43) Pub. Date: Sep. 15, 2016

(54) A disposable absorption product for feminine hygiene

(71) Applicant: FuJian NetDragon Websoft Co., Ltd., Fujian (CN)

(72) Inventors: Dejian Liu, Fujian (CN); Minxin Ma, Fujian (CN); Xuehua Huang, Fujian (CN)

(21) Appl. No.: 14/404,480

(22) PCT Filed: Aug. 6, 2014
(86) PCT No.: PCT/CN2014/083817

§ 371 Date: Nov. 28, 2014

(30) Foreign Application Priority Data
Dec. 17, 2013 (CN) 201320835716.4

(51) Int. Cl. A61B 5/00; A61F 13/84

(52) U.S. Cl. CPC A61B 5/4854; A61B 5/4318; A61F 13/84; A61F 2013/8473

(57) ABSTRACT

The present invention provides a disposable absorption product for feminine hygiene for users to assess their health conditions based on the color of their menstrual blood. The disposable absorption product for feminine hygiene comprises a surface layer facing to a human body, a bottom layer facing away from the human body and an absorptive layer disposed between the surface layer and the bottom layer for absorbing liquid; the bottom layer or the surface layer comprises at least one blood color comparison zone which has a color difference compared with zones outside of its peripheries. Compared with the existing prior art, the present invention provides at least one blood color comparison zone on an outer surface of the absorption product for users to observe. By comparing the color of the menstrual blood on the absorption product and the color of the at least one blood color comparison zone, users can assess their corresponding health conditions. The present invention enables fast, convenient and hygienic assessment of health condition at a low cost.

### BACKGROUND OF THE INVENTION

The present invention relates to the field of absorption products for feminine hygiene, and more specifically relates to a disposable absorption product for feminine hygiene.

A disposable absorption product for feminine hygiene, for example a feminine sanitary napkin (or feminine sanitary pad), is a kind of absorptive product mainly made by cotton, nonwoven fabric, paper pulp, or high molecular polymer/high molecular polymer composite paper made from a compound of the above materials.

A feminine sanitary napkin usually comprises a surface layer facing to the human body, a bottom layer facing away from the human body, and an absorptive layer disposed between the surface layer and the bottom layer for absorbing liquid. Wings are designed mainly to prevent leakage at two sides. The bottom layer of the feminine sanitary napkin is impermeable so that menstrual blood discharged from vagina during feminine periods can be retained and absorbed within the feminine sanitary napkin.

Nowadays, consumers do not simply purchase feminine sanitary napkins for their basic purpose of maintaining feminine hygiene. Instead, consumers are also looking for what other purposes feminine sanitary napkins can serve. Normal menstruation reflects healthy condition. In Chinese medical theories about vital energy (known as “Qi” in the field of Chinese medicine) and blood (known as “Xue” in the field of Chinese medicine), sufficiency of vital energy, healthiness of blood and thus a general health condition can be preliminarily determined from the color of menstrual blood. Menstrual blood having light color is generally associated with the problem of deficiency (known as “Xu” in the field of Chinese medicine), for example deficiency in vital energy and blood, weak spleen and kidney, or occurrence of phlegm and dampness. Menstrual blood having dark color such as crimson, dull red, purplish red, purplish black or ink black is generally associated with the problems of repletion (known as “Shi” in the field of Chinese medicine), hotness (known as “Re” in the field of Chinese medicine), stagnation (known as “Yu” in the field of Chinese medicine) and clogging (known as “Du” in the field of Chinese medicine). Different colors of blood can reflect different health conditions. Different numeral values can be assigned to different colors so that different health conditions can be directly reflected by the numeral values. However, in the absence of a device for color comparison during direct color observation of the menstrual blood on the feminine sanitary napkin for health assessment, it is not easy to determine what the color is and thereby easily resulting in incorrect assessment.

### BRIEF SUMMARY OF THE INVENTION

In view of the aforesaid disadvantages now present in the prior art, the present invention provides a disposable absorption product for feminine hygiene for users to assess their health conditions based on the color of their menstrual blood.

To attain the above object, the present invention provides a disposable absorption product for feminine hygiene comprising a surface layer facing to a human body, a bottom layer facing away from the human body and an absorptive layer disposed between the surface layer and the bottom layer for absorbing liquid.

The bottom layer or the surface layer comprises at least one blood color comparison zone. The at least one blood color comparison zone has a color difference compared with zones outside peripheries of the at least one blood color comparison zone.

Preferably, there are more than two blood color comparison zones, and the more than two blood color comparison zones have color differences.

Preferably, the at least one blood color comparison zone has a color of human blood.

Preferably, the at least one blood color comparison zone is a color strip or a color patch attached to or printed on the bottom layer or the surface layer.

More preferably, the color strip or the color patch is laminated by compression or adhered onto the bottom layer or the surface layer.

Preferably, the at least one blood color comparison zone is disposed at a front edge or at a side edge of the surface layer.

More preferably, the surface layer and the bottom layer of the disposable absorption product for feminine hygiene each has a shape comprising a main body at a center portion and two wings at two sides. The at least one blood color comparison zone is positioned at a side edge of the surface layer, particularly being positioned on the wings on the surface layer.

Preferably, the surface layer has a color of white, beige, pink or light green. The at least one blood color comparison zone is disposed on the surface layer.

Compared with the existing prior art, the present invention provides at least one blood color comparison zone on an outer surface of the absorption product for users to observe. By comparing the color of the menstrual blood on the absorption product and the color of the at least one blood color comparison zone, users can assess their corresponding health conditions. The present invention enables fast, convenient and hygienic assessment of health condition at a low cost.

### BRIEF DESCRIPTION OF THE DRAWINGS

FIG. 1 is a structural view of a disposable absorption product for feminine hygiene according to an embodiment of the present invention.

### REFERENCE SIGNS

1: Feminine sanitary napkin
10: main body
11: front end of surface layer
12: one of the wings
13: another one of the wings
14: rear end of surface layer
21: first color strip
22: second color strip
23: third color strip

### DETAILED DESCRIPTION OF THE INVENTION

In order to explain the technical proposal, the structural characteristics, the objects and the effects of the present invention, the present invention is further described below with reference to an embodiment and the accompanying drawings.

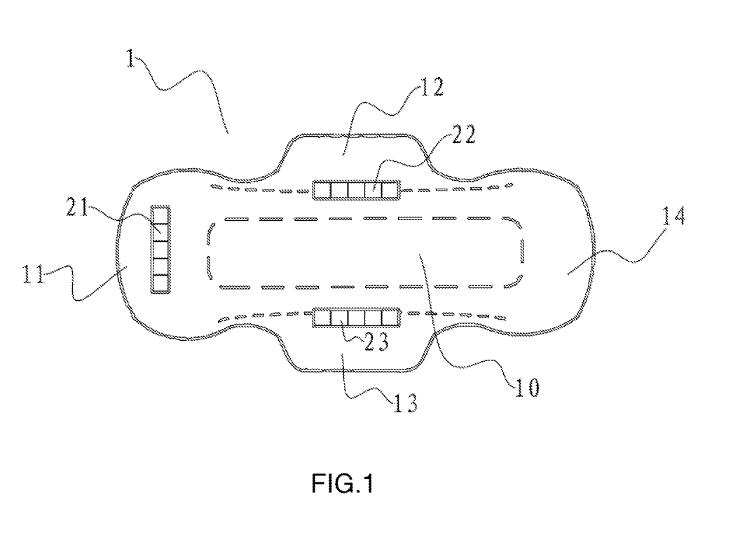

With reference to FIG. 1, the present invention provides a disposable absorption product for feminine hygiene in form of a feminine sanitary napkin 1. The disposable absorption product for feminine hygiene comprises a surface layer facing to a human body, in particular, the surface layer contacts with a vulva of the human body. The disposable absorption product for feminine hygiene also comprises a bottom layer facing away from the human body and an absorptive layer disposed between the surface layer and the bottom layer for absorbing liquid. FIG. 1 shows a structure of the surface layer.

The bottom layer or the surface layer comprises at least one blood color comparison zone. The at least one blood color comparison zone has a color difference compared with zones outside peripheries of the at least one blood color comparison zone. In the present embodiment, the at least one blood color comparison zone is provided on the surface layer. As such, the at least one blood color comparison zone is provided on the same surface as where the menstrual blood is discharged, thereby allowing convenient observation.

Commonly speaking, the surface layer or the bottom layer, in particular the surface layer, of the disposable absorption product for feminine hygiene, has a color of white, beige, pink or light green. In order that users can easily observe and compare the difference and the degree of difference between the color of the menstrual blood on the surface layer and the color of the at least one blood color comparison zone and to ensure convenient and accurate visual comparison, the at least one blood color comparison zone is made with a color difference compared with its surrounding zones, which are the zones outside the peripheries of the at least one blood color comparison zone. In the present embodiment, the at least one blood color comparison zone has a color of human blood. In the present embodiment and as shown in FIG. 1, there are three blood color comparison zones. As shown in FIG. 1, the three blood color comparison zones comprise a first color strip 21, a second color strip 22 and a third color strip 23. The three blood color comparison zones have color differences, in other words, the three blood color comparison zones have three respective different colors, and the three colors are associated with three respective colors of menstrual blood according to three different physiological conditions. The three different colors can be different in terms of hue or tone. Provision of more colors can allow users to correspond the colors of their menstrual blood to the corresponding physiological or health conditions more quickly.

In the present invention, the three blood color comparison zones are color strips attached to the bottom layer or the surface layer. In some other embodiments, each of the blood color comparison zones can be a color strip or a color patch printed on the bottom layer or the surface layer. For more secured attachment onto the surface layer or the bottom layer, the color strip or the color patch is laminated by compression or adhered onto the bottom layer or the surface layer.

In the present invention, the feminine sanitary napkin 1 has wings. In particular, when the surface layer or the bottom layer faces towards the front and the feminine sanitary napkin 1 is viewed from the front, the surface layer and the bottom layer each has a shape comprising a main body 10 at a center portion and two wings 12, 13 at two sides. The wings 12, 13 extend out of the two sides of the main body 10 such that the feminine sanitary napkin 1 has a cross shape. The first color strip 21 is positioned near a periphery of a front end 11 of the surface layer. The second color strip 22 and the third color strip 23 are positioned respectively at two side edges of the surface layer, thereby being positioned on the wings on the surface layer. In some other embodiments, the color strips can also be positioned at a rear end 14 of the surface layer. Since the color strips are disposed at a front edge or at the side edges of the surface layer, the color strips are disposed at positions facilitating convenient observation and they are not easy to be contaminated by the menstrual blood.

By simple color comparison, users are able to know and assess from the color of their menstrual blood whether their health conditions fall within a range defining normal healthy conditions. Therefore, the present invention enables users to timely and conveniently monitor their health conditions.

The above detailed description is intended to describe only an embodiment of the present invention but not intended to limit the scope of the present invention. Application of any changes in terms of configuration or structure made in accordance with the disclosure of the present invention to any related field of art should also fall within the scope of protection of the present invention.

What is claimed is:

1. A disposable absorption product for feminine hygiene, wherein the disposable absorption product for feminine hygiene comprises a surface layer facing to a human body, a bottom layer facing away from the human body and an absorptive layer disposed between the surface layer and the bottom layer for absorbing liquid;
the bottom layer or the surface layer comprises at least one blood color comparison zone; the at least one blood color comparison zone has a color difference compared with zones outside peripheries of the at least one blood color comparison zone.

2. The disposable absorption product for feminine hygiene as in claim 1, wherein the at least one blood color comparison zone comprises more than two blood color comparison zones, and the more than two blood color comparison zones have color differences.

3. The disposable absorption product for feminine hygiene as in claim 1, wherein the at least one blood color comparison zone has a color of human blood.

4. The disposable absorption product for feminine hygiene as in claim 1, wherein the at least one blood color comparison zone is a color strip or a color patch attached to or printed on the bottom layer or the surface layer.

5. The disposable absorption product for feminine hygiene as in claim 4, wherein the color strip or the color patch is laminated by compression or adhered onto the bottom layer or the surface layer.

6. The disposable absorption product for feminine hygiene as in claim 1, wherein the at least one blood color comparison zone is disposed at a front edge or at a side edge of the surface layer.

7. The disposable absorption product for feminine hygiene as in claim 6, wherein the surface layer and the bottom layer of the disposable absorption product for feminine hygiene each has a shape comprising a main body at a center portion and two wings at two sides; the at least one blood color comparison zone is positioned at the side edge of the surface layer, particularly on one of the wings on the surface layer.

8. The disposable absorption product for feminine hygiene as in claim 1, wherein the surface layer has a color of white, beige, pink or light green; the at least one blood color comparison zone is disposed on the surface layer.
